(19) United States
(12) Patent Application Publication
(10) Pub. No.: US 2019/0255127 A1
(43) Pub. Date: Aug. 22, 2019

(54) Augmentation of Oncolytic Viral Efficacy through Immunological Targeting Tumor Endothelial Cells

(71) Applicant: BATU BIOLOGICS, INC., San Diego, CA (US)

(72) Inventors: Samuel C. Wagner, San Diego, CA (US); Thomas E. Ichim, San Diego, CA (US)

(21) Appl. No.: 16/282,047

(22) Filed: Feb. 21, 2019

Related U.S. Application Data
(60) Provisional application No. 62/633,191, filed on Feb. 21, 2018.

(51) Int. Cl. A61K 35/763; A61P 35/00; A61K 9/00; A61K 35/768; A61K 35/765; A61K 35/44; C12N 5/071; A61K 39/00

(52) U.S. Cl. CPC A61K 35/763; A61P 35/00; A61K 9/0019; A61K 35/768; A61K 2039/54; A61K 35/44; C12N 5/069; A61K 39/0011; A61K 35/765

(57) ABSTRACT

Disclosed are means of treatment of cancer by enhancing efficacy of oncolytic virus ability to eradicate tumors through the destruction/inactivation of cancer endothelial cells through immunological means. In one embodiment of the invention, administration of placental endothelial cells generated antitumor endothelial immune responses are used to sensitize tumors to oncolytic viral entry. In another embodiment, oncolytic viruses are utilized to enhance generation of cancer endothelial specific responses by causing localized inflammation in the tumor endothelium, which enhances efficacy of the tumor endothelial targeting vaccine. In another embodiment, the invention teaches the use of replication deficient oncolytic viruses to deliver proteins to tumor cells in an immunogenic manner such that proteins encoded by the oncolytic viruses induce immunity to tumor endothelial cell antigens.

### CROSS-REFERENCE TO RELATED APPLICATIONS

This Patent Application takes priority from Provisional Patent Application No. 62/633,191, titled Augmentation of Oncolytic Viral Efficacy through Immunological Targeting Tumor Endothelial Cells, filed on Feb. 21, 2018, the contents of which are expressly incorporated herein by this reference as though set forth in their entirety and to which priority is claimed.

### BACKGROUND OF THE INVENTION

Oncolytic viruses (OVs), either naturally occurring or evolved and engineered for cancer specificity, are gaining momentum as a new drug class in the fight against cancer. Besides, causing the death of virus-infected cancer cells, the spreading intratumoral (IT) infection can also boost the anticancer immune response, leading to immune destruction of uninfected cancer cells. The key desirable characteristics of any OV are specificity, potency and safety; specificity for the targeted cancer, potency to kill infected cells and cross-prime antitumor immunity, and safety to avoid adverse reactions and pathogenic reversion.

Excitement around the use of oncolytic viruses comes from studies that have, by now, well established in several rodent cancer models that a single dose of an effective OV can completely cure disease. For example, Naik et al demonstrated that oncolytic vesicular stomatitis virus can be engineered to minimize its neurotoxicity, enhance induction of antimyeloma immunity and facilitate noninvasive monitoring of its intratumoral spread. Using high-resolution imaging, autoradiography and immunohistochemistry, they demonstrated that the intravenously administered virus extravasates from tumor blood vessels in immunocompetent myeloma-bearing mice, nucleating multiple intratumoral infectious centers that expand rapidly and necrose at their centers, ultimately coalescing to cause extensive tumor destruction. This oncolytic tumor debulking phase lasts only for 72 h after virus administration, and is completed before antiviral antibodies become detectable in the bloodstream. Antimyeloma T cells, cross-primed as the virus-infected cells provoke an antiviral immune response, then eliminate residual uninfected myeloma cells. The study establishes a curative oncolytic paradigm for multiple myeloma where direct tumor debulking and immune eradication of minimal disease are mediated by a single intravenous dose of a single therapeutic agent. In another study, Yu et al. developed a replication-competent vaccinia virus, GLV-1h68, designed to specifically target human pancreatic carcinomas in cell cultures and in nude mice. They found that GLV-1h68 was able to infect, replicate in, and lyse tumor cells in vitro. Virus-mediated marker gene expressions were readily detected. Moreover, s.c. PANC-1 pancreatic tumor xenografts were effectively treated by a single i.v. dose of GLV-1h68. Cancer killing was achieved with minimal toxicity. Viral titer analyses in homogenized organs and PANC-1 tumors showed that the mutant virus resides almost exclusively in the tumors and not in healthy organs. Except mild spleen enlargements, no histopathology changes were observed in any other organs 2 months after virus injection. Surprisingly, s.c. MIA PaCa-2 pancreatic tumors were treated with similar efficiency as PANC-1 tumors, although they differ significantly in sensitivity to viral lysis in cell cultures. When GLV-1h68 oncolytic viral therapy was used together with cisplatin or gemcitabine to treat PANC-1 tumors, the combination therapy resulted in enhanced and accelerated therapeutic results compared with the virus treatment alone. Profiling of proteins related to immune response revealed a significant proinflammatory immune response and marked activation of innate immunity in virus-colonized tumors.

Studies have shown for DNA and RNA viruses in diverse tumor models that potent tumor killing is feasible. However, while the single shot cure is an exciting prospect for cancer therapy, to date clinical outcomes have typically fallen short of this, and repeat IT virus administration has proven to be a more reliable approach. But there are a number of anecdotal case reports that give credence to the idea that a single shot OV cure may be achievable in clinical practice suggesting that OVs have the potential to transform the practice of oncology. Given the recent clinical progress, interest in the approach is burgeoning. One critical milestone was the 2015 marketing approval granted in Europe and the USA for talimogene laherparepvec (T-Vec, Imlygic™), an engineered HSV encoding GM-CSF. This virus, administered intratumorally every 2 weeks for malignant melanoma, led to complete resolution in 47% of injected tumors and boosted systemic antitumor immunity leading to resolution of 9% of distant uninfected visceral tumors. Despite these advancements, responses in the clinical still range from 10-20% of patients. New treatments are desirable, and/or means of augmenting efficacy of oncolytic virus effects.

### DESCRIPTION OF THE INVENTION

In one embodiment the virus is engineered to encode multiple proteins for expression on the surface of the infected cancer cell wherein at least one protein encoded is an angiogenic protein found on tumor endothelial cells. The angiogenic associated proteins are selected from a group comprising of TEM-1, CD105, VEGF-R, EGF-R, ROBO family members, PDGF-receptor, and angiopoietin receptor. In one embodiment, the VEGF-R or an active fragment thereof, for example two, three, four or more different proteins are encoded, in particular two or three proteins are encoded by the virus for expression on the cancer cell surface or secretion into the extracellular space. Protein in this context includes a fusion protein. In one embodiment the virus of the present disclosure encodes two different VEGF-R proteins, active fragments thereof or combinations of the same, for example both for expression on a cancer cell surface. In one embodiment the virus according to the present disclosure encodes one or two proteins for cell surface expression and one or two proteins which are not capable of being anchored on the cell surface, for example that are intended to act with the cancer cell or are for secretion/release from the cells. In one embodiment a protein associated with tumor blood vessels or active fragment is encoded by the virus of the present disclosure for expression on the surface of the cancer cell and a soluble form, which is released or secreted from the cell, of the same protein or a different protein (including active fragments) is also encoded by the virus.

In one embodiment the multiple proteins may be encoded to be expressed as separate proteins which are independently processed and expressed in the cancer cell membrane. The independence of the proteins on the surface of the cancer cell may make a positive contribution to the immune activation. Whilst not wishing to be bound by theory, lipid packing can influence the fluidity (i.e. the viscosity) of the lipid bilayer in the membrane of the cancer cell. Viscosity of the membrane can affect the rotation and orientation of proteins and other bio-molecules within the membrane, thereby affecting the functions of these molecules. Thus when the proteins encoded by the virus are located as individual and separate proteins within the membrane of the infected cancer cell, the fluidity of the lipid bilayer allows independent movement of the molecules which may be a particularly suitable format, for example similar to a natural format that is conducive to biological function.

In one embodiment the oncolytic virus is used to transduce a suicide gene, such as cytosine deaminase::uracil phosphoribosyltransferase (CD::UPRT), to convert the relatively nontoxic 5-fluorocytosine (5-FC) into the highly toxic antitumor 5-fluorouracil (5-FU). In other embodiments, mesenchymal stem cells are used for delivery of oncolytic or tumor trophic viruses, including adenoviruses, vaccinia virus, herpes virus, reovirus, measles, Newcastle Disease Virus. The viruses may be used alone without delivery by cells.

In one embodiment the independently processed and expressed proteins are located (anchored) in different locations, such as physically separate locations, in the cancer cell membrane. In one embodiment one or more proteins (for example all the proteins) encoded by the virus and expressed on the surface of the infected cancer cell are not fusion proteins.

As described above in some embodiment the proteins are expressed as a fusion protein.

In one embodiment the virus of the present disclosure provides one or more separate independent proteins for cell surface expression and one or more fusion proteins for cell surface expression. Thus, in one embodiment, the virus according to the present disclosure comprises DNA sequences encoding the multiple proteins for expression, for example on the surface or the infected cancer cell.

In some embodiments of the invention, cytokines, which are known in the art to possess tumor inhibitory properties are transfected using either the virus itself, combinations of the virus and cell mediated delivery, or directly into the tumor. The cytokines include TRAIL, TNF-alpha, interferon gamma, interferon alpha, interferon beta, IL-12, IL-18, IL-21, and IL-28.

Thus, in one embodiment, the virus according to the present disclosure comprises two or more transgenes, in the same or different locations in the virus genome. When located at the same position in the virus genome the multiple proteins will still be expressed independently at the surface of the cancer cell. In one embodiment the multiple proteins (including fusion proteins) are encoded in the same location in the virus genome and expressed together on the infected cancer cell surface, for example where the proteins encoded are provided as a fusion protein, in particular wherein the fusion protein comprises an angiogenic protein associated with the tumor vasculature or an active fragment thereof. Specific proteins include TEM-1, CD105, VEGF-R, EGF-R, ROBO family members, PDGF-receptor, and angiopoietin receptor. In one embodiment costimulatory proteins such as the B7 protein in the fusion protein is a full-length protein, in particular a protein described herein, such as B7-1 and/or B7-2, fused or linked to another protein of interest or an active fragment thereof. In one embodiment, the fusion protein comprises a transmembrane from a B7 protein. In one embodiment the B7 is an active fragment excluding the transmembrane domain. In the latter embodiment a transmembrane other than one derived from a B7 protein may be employed to ensure the fusion protein is presented on the surface of the infected cancer cell. In one embodiment the multiple proteins are encoded in the same location in the virus and are expressed as one or more fusion proteins together on the surface of the infected cancer cell. Thus in one embodiment the virus encodes B7-1, B7-2 or an active fragment of any one of the same or a combination thereof for expression on the surface of the infected cancer cell and an anti-CD3 (agonist) antibody or antibody binding fragment (such as a scFv) also for expression on the cancer cell surface (in particular where the proteins are expressed as individual proteins on the cell surface) and further encodes a cytokine independently selected from IL-2, IFN-alpha., IFN-gamma., GM-CSF, IL-15, and IL-12, and or a chemokine selected from RANTES (CCL5), MIP1-alpha. (LD78a (CCL3) or LD78-beta (CCL3L1) isoforms), MIP1-beta which can be released from the cancer cell, in particular by secretion before and release after cell lysis/death of the infected cancer cell.

In one embodiment of the invention, when the location of the gene(s) encoding a protein or protein(s) of interest in the virus is the same then the genes may, for example be linked by an IRES sequence or a 2A peptide. In one embodiment the virus according to the present disclosure comprises a “second” transgene and optionally a third transgene (i.e. one or more of the multiple proteins, for example encoding a polypeptide selected from the group comprising a cytokine, a chemokine, a ligand, and an antibody molecule, such as an antagonistic antibody molecule, and an agonistic antibody molecule. In one embodiment the additional protein or proteins is/are independently selected from the group comprising an antibody, antibody fragment or protein ligand that binds CD3, CD28, CD80, CD86, 4-1BB, GITR, OX40, CD27, CD40 and combinations, for example in forms suitable for expression on the surface of a cancer cell.

Optimal T cell activation requires simultaneous signals through the T cell receptor and costimulatory molecules. The costimulatory molecule CD28, upon interaction with its ligands B7-1 and B7-2, plays a crucial role in initial T cell priming. However, the CD28-mediated T cell expansion is opposed by the B7-1/2 counter receptor, cytotoxic T lymphocyte associated antigen 4 (CTLA-4), which mitigates the proliferation of recently activated T cells. This sequential regulation of CD28 and CTLA-4 expression balances the activating and inhibitory signals and ensures the induction of an effective immune response, while protecting against the development of autoimmunity. Blocking of CTLA-4 with monoclonal antibodies has demonstrated some success in human clinical trials. Additional CD28 and B7 family members have been identified: PD-1 (programmed death-1), PD-L1 (programmed death ligand-1 or B7-H1), and PD-L2 (B7-DC). As in the CTLA-4/B7 system, the PD-1 interactions with PD-L1 and PD-L2 suppress both central and peripheral immune responses, and therefore, the PD-1 blockade is also being explored in clinical trials. In addition, numerous new agents targeting the inhibitory and activation pathways involved in T-cell modulation such as LAG-3, B7-H3, CD40, OX40, CD137 and others are in active development.

Accordingly, in some embodiments, T-cell induction comprises administration an agonist of an activating co-stimulatory molecule. In some embodiments, the method comprises administration of agonistic antibodies directed against activating co-stimulatory molecules. In some embodiments, T-cell induction comprises administration of agonistic antibodies against a co-stimulatory molecule selected from the group consisting of: CD28, OX40, GITR, CD137, CD27 and HVEM.

In some embodiments, T-cell induction comprises administration of a treatment that antagonizes negative co-stimulatory molecules. In some embodiments, the method comprises administration of blocking antibodies against negative co-stimulatory molecules. In some embodiments, T-cell induction comprises administration of blocking antibodies against a negative co-stimulatory molecule selected from the group consisting of: CTLA-1; PD-1, TIM-3, BTLA, VISTA and LAG-3. In some embodiments, T-cell induction comprises administration of CTLA-4 blocking antibodies. In some embodiments, T-cell induction comprises administration of PD-1 pathway inhibitors. In some embodiments, the inhibitor of the PD-1 pathway is selected from antibodies against PD-1 and soluble PD-1 ligand. In some embodiments, the inhibitors of the PD-1 pathway are selected from AMP-244, MEDI-4736, MPDL328 OA, and MIH1.

In some embodiments, T-cell induction comprises administration of a treatment that stimulates T-cell expansion. In some embodiments, a treatment that stimulates T-cell expansion comprises administration of cytokines. In some embodiments, a treatment that stimulates T-cell expansion comprises administration of cytokine-inducing viruses.

Other proteins may be added to the viral construct that stimulate T cells. For example, in one embodiment the additional protein is an anti-CD3 antibody, for example independently selected from a Muromonab-CD3 (also known as OKT3), otelixizumab (also known as TRX4), teplizumab (also known as hOKT3.gamma.1 (Ala-Ala)), or visilizumab. According to known techniques in the art, in one embodiment the anti-CD3 antibody is in the form of an antibody fragment, for example an scFv that is part of a fusion protein with the transmembrane region of another protein, for example the transmembrane domain from the PDGF receptor or from the cell surface form of IgG. In one embodiment an antibody molecule is an inhibitor (antagonistic antibody) is independently selected from the group comprising an inhibitor of an angiogenesis factor, such as an anti-VEGF antibody molecule, and inhibitor of T cell deactivation factors, such as an anti-CTLA-4, anti-PD1 or anti-PDL1 antibody molecule. In one embodiment antibody molecule is an agonist independently selected from the group comprising antibodies to CD40, GITR, OX40, CD27 and 4-1BB.

Immunotherapeutic molecules such as bispecific antibodies may be utilized to activate immune cells in the proximity of the tumor. Immunoapoptins may also be used for transfection.

The nucleic acid molecule for use in the methods of the presently disclosed subject matter may encode one or more bioactive molecules functional in the treatment of an oncological indication. The one or more bioactive molecules may be selected from the group consisting of proteins, polypeptides, peptides, drugs, enzymes, hormones, RNA, and metabolites. In a particular embodiment, the cancer is a brain tumor, and the one or more bioactive molecules comprise one or more anti-cancer agents, particularly wherein the one or more anti-cancer agents are selected from the group consisting of bone morphogenic protein 4 (BMP4), TNF-related apoptosis-inducing ligand (TRAIL), HSV-thymidine kinase, an oncolytic adenovirus, interleukin-2 (IL-2), interleukin-12 (IL-12), interleukin-18 (IL-18), interleukin-23 (IL-23), Interferon-alpha, and Interferon-beta.

The nucleic acid molecule for use in the methods of the presently disclosed subject matter may encode one or more bioactive molecules functional in the treatment of a neurological disease. The one or more bioactive molecules may be selected from the group consisting of proteins, polypeptides, peptides, drugs, enzymes, hormones, RNA, and metabolites. In a particular embodiment, the neurological disease is a brain tumor, and the one or more bioactive molecules comprise one or more anti-cancer agents, particularly wherein the one or more anti-cancer agents are selected from the group consisting of bone morphogenic protein 4 (BMP4), TNF-related apoptosis-inducing ligand (TRAIL), HSV-thymidine kinase, an oncolytic adenovirus, interleukin-2 (IL-2), interleukin-12 (IL-12), interleukin-18 (IL-18), interleukin-23 (IL-23), Interferon-alpha, and Interferon-beta.

In one embodiment an additional transgene encodes a cytokine, or soluble variant thereof selected from the group comprising IL-2, IFN-alpha, IFN-beta, IFN-gamma, GM-CSF, IL-15, IL-12 and fms-related tyrosine kinase 3 ligand (FLT3L). Advantageously, one or more of this group of proteins expressed by the virus, in particular as a free protein secreted from the cancer cell, may be particularly suitable for stimulating an immune response in vivo to the cancer cell. In one embodiment an additional transgene encodes a chemokine, selected from the group comprising MIP1-alpha, IL-8, CCL5, CCL17, CCL20, CCL22, CXCL9, CXCL10, CXCL11, CXCL13, CXCL12, CCL2, CCL19 and CCL21. Advantageously, one or more of this group of proteins is expressed by the virus as a free protein which may be secreted from the cancer cell may be particularly suitable for attracting immune cells and stimulating an immune response to the cancer cell in vivo. In one embodiment in addition to at least the B7 protein or active fragment thereof expressed on the surface of the infected cancer cell, one or more molecules are also expressed on the surface and/or secreted.

The practice of one embodiment of the invention is directed to combination therapies including administering a chemotherapeutic drug with a nucleic acid composition useful as an immunogen for triggering immunity towards the tumor endothelium, comprising a combination of: (a) first nucleic acid vector comprising a first sequence encoding an antigenic polypeptide or peptide, which first vector optionally comprises a second sequence linked to the first sequence, which second sequence encodes an immunogenicity-potentiating polypeptide (IPP); b) a second nucleic acid vector encoding an anti-apoptotic polypeptide, wherein, when the second vector is administered with the first vector to a subject, a T cell-mediated immune response to the antigenic polypeptide or peptide is induced that is greater in magnitude and/or duration than an immune response induced by administration of the first vector alone. The first vector above may comprise a promoter operatively linked to the first and/or the second sequence.

For the purpose of the invention, oncolytic viruses not only comprise a class of vectors able to encode and express a particular antigen to which an antigen-specific immune response is desired, but it also mediates killing of cancer cells. The term “oncolytic” and “oncolytic viruses” refer to cancer killing, i.e. “onco” meaning cancer and “lytic” meaning “killing”. As used herein, where oncolytic refers to an “oncolytic virus” and an “OV,” this virus represents a virus that may kill a cancer cell. In principle any virus capable of selective replication in neoplastic cells including cells of tumors, neoplasms, carcinomas, sarcomas, and the like may be utilized in the invention. Selective replication in neoplastic cells means that the virus replicates at least 1×10⁴, 1×10⁵, 1×10⁶, or more efficient in at least three cell lines established from different tumors compared to cells from at least three different non-tumorigenic tissues. Oncolytic viruses may additionally or alternatively be targeted to specific tissues or tumor tissues. This can be achieved for example through transcriptional targeting of viral genes (e.g. WO 96/39841, incorporated by reference) or through modification of viral proteins that are involved in the cellular binding and uptake mechanisms during the infection process (e.g. WO 2004033639 or WO 2003068809, all of which are incorporated by reference).

A wide variety of herpes viruses, Adenovirus, Adeno-associated virus, influenza virus, reovirus, vesicular stomatitis virus (VSV), Newcastle virus, vaccinia virus, poliovirus, measles virus, mumps virus, sindbis virus (SrN) and sendai virus. To provide more information for one of skill in the art practicing the invention, a description is provided of some of the viruses possessing oncolytic properties that render the viruses useful for the practice of the invention. Oncolytic adenoviruses are double-stranded DNA viruses. While non-replicating adenoviruses have been extensively used as gene therapy vectors, replicating adenoviruses have been engineered to be tumor-specific agents. These tumor-targeting properties of adenoviruses have been engineered in three ways: deletion of critical viral genes; insertion of tumor/tissue-specific promoters; and modification of the viral fiber knob used for cell entry. The prototypical tumor-selective replicating adenovirus is ONYX 015, in which the E1B 55K gene was deleted. ONYX-015 causes tumor-specific cytolysis and antitumoral efficacy that can be augmented by standard chemotherapeutic agents.

Measles virus, a member of the paramyxoviridae family, is a negative strand RNA virus. While the wild-type measles virus is a human pathogen, the vaccine strain Edmonston B (MV-Edm) is highly attenuated in normal human cells. Despite this attenuation, MV-Edm is a potent oncolytic virus. Vesicular stomatitis virus, VSV, is a small, negative strand, RNA virus of the rhabdoviridae family. While it naturally has a wide tissue tropism, it causes a very mild infection in humans, perhaps due to its unique sensitivity to IFN. Phosphorylation of double-stranded RNA-activated protein kinase (PKR) and induction of IFN-responsive genes in normal cells is a critical antiviral response to VSV infection. Several mutant VSVs that induced IFN production have been described. This resulted in increased protection of mice infected with the mutant VSV compared with the wild type virus thus improving the safety profile of these viruses. As many cancer cells have defects in their IFN pathways, they have been shown to be supportive of a productive VSV infection and hence selectively killed. VSV has previously been shown to selectively replicate and kill tumors with aberrant p53, ras or myc signalling accounting for up to 90% of cancers. Reovirus is a double-stranded RNA virus belonging to the reoviridae family. It causes no known pathology in humans making it an ideal candidate for oncolytic virotherapy. Reovirus was discovered to have oncolytic properties when it replicated preferentially in cancer cells with activated ras pathways.

Coxsackievirus A21 (CAV21) has been shown to have oncolytic activity in melanoma and recently multiple myeloma. CAV21 is a positive-strand RNA virus and a member of the picornaviridae family. CAV21 is one agent responsible for ‘common-cold’ symptoms in man but has caused no major disease. The tumor-specificity of CAV21 is through its binding to two cellular receptors: intercellular adhesion molecule 1 (ICAM-1) and decay-accelerating factor (DAF), both upregulated in human tumors compared with normal tissues. Thus in one embodiment the virus encodes B7-1, B7-2 or an active fragment of any one of the same or a combination thereof. Thus, in one embodiment the virus encodes B7-1, B7-2 or an active fragment of any one of the same or a combination thereof for expression on the surface of the infected cancer cell and an anti-CD3 (agonist) antibody or antibody binding fragment (such as a scFv) also for expression on the cancer cell surface, in particular where the proteins are expressed as individual proteins on the cell surface.

Thus, in one embodiment the virus encodes B7-1, B7-2 or an active fragment of any one of the same or a combination thereof for expression on the surface of the infected cancer cell and an anti-VEGF (antagonist) antibody or a binding fragment thereof also for expression on the cancer cell surface or for release from the cancer cell, for example by secretion or after lysis/death of the infected cancer cell.

Thus, in one embodiment the oncolytic virus encodes B7-1, B7-2 or an active fragment of any one of the same or a combination thereof for expression on the surface of the infected cancer cell and an antibody, antibody fragment or protein ligand that binds CD3, CD28, CD80, CD86, 4-1BB, GITR, OX40, CD27, CD40 also for expression on the cancer cell surface or for release from the cancer cell, for example by secretion or release after lysis/death of the infected cancer cell. Furthermore, in one embodiment the virus encodes B7-1, B7-2 or an active fragment of any one of the same or a combination thereof for expression on the surface of the infected cancer cell and a cytokine selected from IL-2, IFN-alpha, IFN-beta, IFN-gamma, GM-CSF, IL-15, IL-12, and FLT3L, for example for release from the cancer cell, in particular by secretion or release after cell lysis/death of the infected cancer cell. Thus in one embodiment the virus encodes B7-1, B7-2 or an active fragment of any one of the same or a combination thereof for expression on the surface of the infected cancer cell and an anti-CD3 (agonist) antibody or antibody binding fragment (such as a scFv) also for expression on the cancer cell surface (in particular where the proteins are expressed as individual proteins on the cell surface) and further encodes a cytokine or chemokine selected from IL-2, IFN-alpha, IFN-gamma, GM-CSF, IL-15, IL-12, FLT3L, MIP1-alpha, IL-8, CCL5, CCL17, CCL20, CCL22, CXCL9, CXCL10, CXCL11, CXCL13, CXCL12, CCL2, CCL19, CCL21 for example for release from the cancer cell, in particular by secretion or after cell lysis/death of the infected cancer cell. Furthermore, the virus encodes B7-1, B7-2 or an active fragment of any one of the same or a combination thereof for expression on the surface of the infected cancer cell and an anti-CD3 (agonist) antibody or antibody fragment (such as a scFv) also for expression on the cancer cell surface (in particular where the proteins are expressed as individual proteins on the cell surface) and further encodes an antibody, antibody fragment or protein ligand that binds CD28, CD80, CD86, 4-1BB, GITR, OX40, CD27, CD40 or an anti-VEGF (antagonist) antibody also for expression on the cancer cell surface or for release from the cancer cell, for example by secretion or release after lysis/death of the infected cancer cell. The virus may encode B7-1, B7-2 or an active fragment of any one of the same or a combination thereof for expression on the surface of the infected cancer cell and two different cytokines or chemokines selected from IL-2, IFN-alpha, IFN-beta, IFN-gamma, GM-CSF, IL-15, and IL-12, FLT3L, MIP1-alpha, IL-8, CCL5, CCL17, CCL20, CCL22, CXCL9, CXCL10, CXCL11, CXCL13, CXCL12, CCL2, CCL19, CCL21, for example for release from the cancer cell, in particular by secretion of after cell lysis/death of the infected cancer cell.

In one aspect of the invention, oncolytic viruses, alone, or in combination with tumor endothelial targeting immunogens, such as ValloVax, are transfected with the nucleic acid molecules may also be administered to the patient in combination with an additional therapeutic agent or treatment. Additional therapeutic agents may also include, but are not limited to, chemotherapeutic agents such as adriamycin, dexamethasone, vincristine, cyclophosphamide, fluorouracil, topotecan, taxol, interferons, and platinum derivatives. Other examples of agents with which the disclosed oncolytic viruses transfected with the nucleic acid molecules may also be administered include, without limitation, anti-inflammatory agents such as corticosteroids, TNF blockers, IL-I RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors, such as acetylcholinesterase inhibitors, MAO inhibitors, interferons, anti-convulsants, and ion channel modifiers.

Examples of use of oncolytic viruses has previously been used in the literature with some success. Means of augmenting efficacy of these approaches by sensitizing the tumor endothelium using vaccination against tumor endothelial, or tumor vascular channels is disclosed. For example, attenuated (nonpathogenic) avian viruses have been used as a form of nonspecific immunological treatment for advanced human cancer. In one study, investigators used Newcastle disease virus (NDV) vaccine MTH-68/N in an open phase II/B, placebo-controlled (26 patients), multicenter clinical trial for the treatment of 33 patients with advanced cancers. NDV (4000 U/day) or placebo was administered by inhalation twice weekly. During the 6-month trial, the size and presence of primary tumors and metastases were objectively monitored at five institutions by radiologists unaware of the type of treatment that was given. Regression of tumor(s) and/or metastases were observed in eight cases treated with virus (vs. none in the placebo group; p<0.01). Ten additional patients treated with NDV had no further progression of their tumor sizes, whereas tumor stabilization was noted in only two control patients. Objective, favorable responses (regressions plus stabilization) to virus therapy thus occurred in a total of 18 patients (55%) compared to 2 patients in the placebo group (8%; p<0.01). Two cases of complete remission were noted in the group treated with NDV. Patients receiving virus therapy had a higher rate of survival at 1 to 2 years. Of 33 patients receiving virus vaccine, 22 survived 1 year, compared to only 4 of 26 patients in the control group (p<0.02). After 2 years, all seven survivors in the study were in the virus therapy group [54]. The invention teaches the augmentation of viral therapy/immunotherapy of cancer by utilization of oncolytic viruses together with tumor vascular targeting immunotherapies.

Although the use of immunotherapy, as described in the invention may be applied in a variety of tumors that are known to possess immunogenic properties, the invention is applicable to numerous types of cancers, which include cancer cells from the anus, bladder, blood, bone, bone marrow, brain, breast, colon, esophagus, gastrointestine, gum, head, kidney, liver, lung, nasopharynx, neck, oral cavity, oropharynx, ovary, penis, prostate, skin, stomach, testis, tongue, cervix, uterus, vagina or vulva. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, chemotherapy is used to reduce tumor volume, and/or increase immunogenicity of tumors. In some embodiments, the treatment that will induce apoptosis in cells within the tumor comprises administration of a chemotherapeutic compound. Chemotherapeutic compounds include, but are not limited to platinum; platinum analogs (e.g., platinum coordination complexes) such as cisplatin, carboplatin, oxaliplatin, DWA2114R, NK121, IS 3 295, and 254-S; anthracenediones; vinblastine; alkylating agents such as thiotepa and cyclosphosphamide; alkyl sulfonates such as busulfan, improsulfan and piposulfan; aziridines such as benzodopa, carboquone, meturedopa and uredopa; ethylenimines and methylamelamines including altretamine, triethylenemelamine, trietylenephosphoramide, triethylenethiophosphaoramide and trimethylolomelamime nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; substituted ureas; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; anti-cancer polysaccharides; polysaccharide-K; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; cytosine arabinoside; cyclophosphamide; thiotepa; taxoids, such as paclitaxel and doxetaxel; chlorambucil; gemcitabine; 6-thioguanine; mercaptopurine; methotrexate; etoposide (VP-16); ifosfamide; mitomycin C; mitoxantrone; vincristine; vinorelbine; navelbine; novantrone; teniposide; daunomycin; aminopterin; XELODA; ibandronate; CPT11; topoisomerase inhibitor RFS 2000; difluoromethylornithine (DMFO); retinoic acid; esperamicins; capecitabine; methylhydrazine derivatives; Erlotinib (TARCEVA); sunitinib malate (SUTENT); and pharmaceutically acceptable salts, acids or derivatives of any of the above.

Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone and toremifene (FARESTON); adrenocortical suppressants; and antiandrogens such as flutamide, nilutamide, bicalutamide, leuprolide and goserelin; and pharmaceutically acceptable salts, acids or derivatives of any of the above. Such chemotherapeutic compounds that can be used herein include compounds whose toxicities preclude use of the compound in general systemic chemotherapeutic methods. In some embodiments, the chemotherapy comprises administration of a chemotherapeutic agent is selected from an alkylating drug, an antimetabolite, an antimytotic cytostatic, a topoisomerase inhibitor, antitumor antibiotic, and any other cytostatic, and/or a radiotherapy. In some embodiments, the chemotherapeutic agent is an alkylating agent. In some embodiments, the alkylating agent is selected from cisplatin, oxaliplatin, cyclophosphamid, ifosfamid, trofosfamid, melphalan, chlorambucil, estramustin, busulfan, treosulfan, carmustin, lomustin, nimustin, streptozocin, procarbazin, dacarbazin, temozolomid, and thiotepa. In some embodiments, the chemotherapeutic agent is an antimetabolite. In some embodiments, the antimetabolite is selected from 5-fluorouracil, methotrexate, azacitidin, capecitabin, doxifluridin, cytarabin, gemcitabin, 6-thioguanin, pentostatin, azathioprin, 6-mercaptopurin, fludarabin, and cladribin. In some embodiments, the chemotherapeutic agent is a topoisomerase inhibitor. In some embodiments, the topoisomerase inhibitor is selected from doxorubicin, camptothecin, topotecan, irinotecan, etoposide, and teniposide. In some embodiments, the chemotherapeutic agent is an antitumor antibiotic. In some embodiments, the antitumor antibiotic is selected from tamoxifen, 5-fluoro-5′-deoxyuridine, belomycin, actinomycin D, and mitomycin. In some embodiments, the chemotherapeutic agent is a cytostatic. In some embodiments, the cytostatic is L-asparaginase or hydroxycarb amide.

In some embodiments, an immune stimulatory virus is utilized to increase interferon production in the proximity of the tumor, or directly in the tumor, so as to sensitize the tumor to killing by tumor vascular targeting approaches. The viruses that are immune stimulatory may be vaccinia virus. In some embodiments increased susceptibility to infection is achieved by culture with histone deacetylase inhibitors such as valporic acid. Subsequent to administration of cells that are virally infected, administration of chemotherapy capable of stimulating immunity is performed. The chemotherapy includes low dose cyclophosphamide administered by various regimens such as metronomic administration in order to decrease T regulatory cells and enhance antitumor immunity. Other agents alone or together with cyclophosphamide may be used such as gemcitabline, everolimus, and doxorubicin.

Accordingly, in some embodiments, in situ vaccination comprises injecting into the subject a modified virus, wherein the modified virus encodes a cytotoxic payload (“Trojan Horse” delivery technology). In some embodiments, the modified virus carries one or more imaging payloads. In some embodiments, the modified virus carries one or more of a virus, an antibody, or a cytokine as the cytotoxic payload. In some embodiments, the modified virus expresses a cytokine as the cytotoxic payload. In some embodiments, the cytokine is selected from colony-stimulating factor (CSF), interferon (IFN), interleukin (IL), stem cell factor (SCF), tumour growth factors (TGF), and tumour necrosis factor (TNF). In some embodiments, the cytokine is a CSF. In some embodiments, the CSF is G-CSF, M-CSF, or GM-CSF. In some embodiments, the CSF is selected from ancestim, garnocestim, pegacaristim, leridistim, milodistim, filgrastim, lenograstim, nartograstim, pegfilgrastim, pegnartograstim, ecogramostim, molgramostim, regramostim, sargramostim, cilmostim, lanimostim, mirimostim, daniplestim, muplestim, or derivates thereof. In some embodiments, the cytokine is an interleukin (IL).

In some embodiments, the interleukin is selected from IL-1 to IL-35, and derivates thereof. In some embodiments, the interleukin is IL-2, IL-4, or derivates thereof. In some embodiments, the cytotoxic payload comprises a lytic virus. In some embodiments, the lytic virus is a vaccinia virus. In some embodiments, the cytotoxic payload comprises a chemotherapeutic agent. In some embodiments, step (b) results in in situ vaccination of the subject against the tumor. Furthermore, in some embodiments of the invention, agents capable of inducing a “danger signal” are administered systemically or intratumorally. Agents that are capable of inducing such an innate immunological response include, a) a TLR agonist; b) intravenous immunoglobulin (IVIG); c) monocyte conditioned media; d) supernatant from neutrophil extracellular trap exposed peripheral blood mononuclear cells; e) co-culture with monocytes; f) co-culture with monocytes that have been pretreated with IVIG; g) co-culture with T cells; h) co-culture with T cells that have been exposed to a T cell stimulus; i) co-culture with NK cells; j) peptidoglycan isolated from gram positive bacteria; k) lipoteichoic acid isolated from gram positive bacteria; l) lipoprotein isolated from gram positive bacteria; m) lipoarabinomannan isolated from mycobacteria, n) zymosan isolated from yeast cell well; o) Polyadenylic-polyuridylic acid; p) poly (IC); q) lipopolysaccharide; r) monophosphoryl lipid A; s) flagellin; t) Gardiquimod; u) Imiquimod; v) R848; w) oligonucleosides containing CpG motifs; and x) 23S ribosomal RNA.

In some embodiments of the invention, stimulation of anticancer response as described by the current invention is performed through combination of anti-angiogenic targeting immunity together with agents known to inhibit angiogenesis. The combination substantially reduces the possibility of treatment resistance. Antiangiogenic agents may be selected from the group consisting of agents that target the vascular endothelial growth factor (VEGF) pathway, an integrin, a matrix metalloproteinase (MMP) and/or protein kinase C beta (PKC-beta), or a combination thereof. In other embodiments inhibition of angiogenesis is accomplished by the receptor antagonist of epidermal growth factor receptor (EGFR) signaling pathway is an EGFR tyrosine kinase inhibitor, in particular wherein the EGFR tyrosine kinase inhibitor is an anti-EGFR monoclonal antibody, more in particular wherein the monoclonal antibody may be selected from a group comprising of cetuximab (Erbitux®), panitumumab (Vectibix®), nimotuzumab, matuzumab, zalutuzumab, mAb 806, or IMC-11F8.

In other embodiments a tyrosine kinase inhibitor is utilized together with an oncolytic virus alone or together with an agent or plurality of agents capable of stimulating immunity towards tumor endothelium. The tyrosine kinase inhibitors are selected from the group consisting of agents that target the vascular endothelial growth factor receptor (VEGFR) pathway, the epidermal growth factor receptor (EGFR) pathway, the platelet-derived growth factor receptor (P1GFR), the fibroblast growth factor receptor (FGFR), ErbB2 or an agent that targets a combination thereof.

In other embodiments, tyrosine kinase inhibitors used for the purpose of the invention target the vascular endothelial growth factor receptor (VEGFR) and are selected from the group consisting of sunitinib (SU11248; Sutent®), SU5416, SU6668, vatalanib (PTK787/ZK222584), AEE788, ZD6474, ZD4190, AZD2171, GW786034, sorafenib (BAY 43-9006), CP-547,632, AG013736, YM-359445, Bevacizumab (Avastin®), 2C3, and HuMV833.

In other embodiments of the invention, agents that target the epidermal growth factor receptor (EGFR) are selected from the group consisting of AEE788, ZD6474, gefitinib (Iressa®), erlotinib (Tarceva®), EKB-569, HKI-272, C1-1033, cetuximab (Erbitux®), panitumumab (Vectibix®), nimotuzumab, matuzumab, zalutuzumab, mAb 806, and IMC-11F8.

In certain embodiments, a nucleic acid composition, such as a DNA vaccine is utilized to induce an immune response to tumor vasculature. The DNA vaccine may include a vector capable of selectively inducing expression of antigens found on tumor vasculature. In some embodiments, the nucleic acid composition is administered from the group consisting of intradermally, intraperitoneally, intravaginally, intramuscularly, subcutaneously, intracervically and intravenously. In certain embodiments, the mammal is a human having a tumor and wherein the nucleic acid composition is administered intratumorally or peritumorally. In some embodiments, the oncolytic virus is selected from the group consisting of vaccinia virus (including Wyeth strain and New York strain, and modified vaccinia virus Ankara), adenovirus, herpes simplex virus, poxvirus, vesicular stomatitits virus, measles virus, Newcastle disease virus, influenza virus, and reovirus. In yet another embodiment, the oncolytic virus is thymidine kinase negative. In certain embodiments, the oncolytic virus is administered from the group consisting of intradermally, intraperitoneally, intracervicovaginally, intramuscularly, subcutaneously and intravenously or into the genital tract or anal cavity or into the oral cavity or oropharynx or a lymph node. In some embodiments, the mammal is a human having a tumor and wherein the oncolytic virus is administered intratumorally or peritumorally. In still other embodiments, the nucleic acid composition is present within an oncolytic virus. In other embodiments, the oncolytic virus of step (a) is the same as or is different from the oncolytic virus of step (b). In yet other embodiments, step (a) is performed before step (b), step (a) and step (b) are performed at the same time, or step (a) is performed after step (b). In still another embodiment, step (a) and/or step (b) is repeated at least once. In one embodiment, the dosage of oncolytic virus used in step (a) and/or step (b) is a range that includes 1×10⁷ pfu.

In one specific embodiment of the invention, a patient suffering from a neoplastic malignancy is treated initially with an immunogenic composition capable of stimulating immunity towards tumor vasculature. Means of generating immunity towards tumor vasculature are known in the art and are described below. Zhuang et al demonstrated that mice immunized with the extracellular domain of mouse Robo4, showed a strong antibody response to Robo4, with no objectively detectable adverse effects on health, including normal menstruation and wound healing. Robo4 vaccinated mice showed impaired fibrovascular invasion and angiogenesis in a rodent sponge implantation assay, as well as a reduced growth of implanted syngeneic Lewis lung carcinoma. The anti-tumor effect of Robo4 vaccination was present in CD8 deficient mice but absent in B cell or IgG1 knockout mice, suggesting antibody dependent cell mediated cytotoxicity as the anti-vascular/anti-tumor mechanism. Another antigen that is more ubiquitously found throughout the body, but with higher expression on tumor endothelial cells is the VEGF receptor 2 (VEGFR2) which is typically found on hematopoietic stem cells and endothelial progenitor cells. Despite expression on non-malignant tissue, successful induction of antitumor immunity has been demonstrated using various immunization means against this antigen. Yan et al utilized irradiated AdVEGFR2-infected cell vaccine-based immunotherapy in the weakly immunogenic and highly metastatic 4T1 murine mammary cancer model. Lethally irradiated, virus-infected 4T1 cells were used as vaccines. Vaccination with lethally irradiated AdVEGFR2-infected 4T1 cells inhibited subsequent tumor growth and pulmonary metastasis compared with challenge inoculations. Angiogenesis was inhibited, and the number of CD8+ T lymphocytes was increased within the tumors. Antitumor activity was also caused by the adoptive transfer of isolated spleen lymphocytes, thus demonstrating induction of tumor specific immunity. Other approaches have been utilized to induce immunity to VEGFR2, which resulted in induction of tumor regression without systemic toxicities. Other approaches have been utilized to induce immunity to VEGFR2, which resulted in induction of tumor regression without systemic toxicities. Tumor endothelial marker 1 or endosialin is another antigen found selectively on the tumor vasculature. Facciponte et al demonstrated that a DNA vaccination targeting endosialin reduced tumor vascularity, increased CD3+ T cell infiltration, and was correlated with significant inhibition of tumor growth. Epitope spreading to tumor antigens following the initial immune response against the tumor vasculature gives evidence that targeting the tumor endothelium may activate a cascade of pathways conducive to tumor regression. Additionally, the DNA vaccination against endosialin did not affect other angiogenesis dependent physiological processes, exhibiting no adverse effects on menstruation, embryonic development, pregnancy, and wound healing in mouse models. Other markers associated with tumor blood vessels have been utilized therapeutically in animal models for vaccination purposes including survivin, endosialin, and xenogeneic FGF2R, VEGF, VEGF-R2, MMP-2, and endoglin.

Administration of agents capable of inducing immunity towards tumor vasculature is performed prior to, concurrently with, or subsequently to administration of oncolytic viruses. In accordance with this invention oncolytic viruses may be administered alone, in various solutions, or together with human cells. The human cells can be derived from any source. Autologous, or allogeneic cells may be used. In an embodiment the human cells comprise leukocytes. The leukocytes utilized in accordance with this invention (e.g. monocytes, neutrophils and lymphocytes including tumor-infiltrating lymphocytes) can be active or inactive. Techniques for inactivating leukocytes include irradiation. The cells utilized in accordance with this invention can be isolated (for example by leukopheresis in the case of leukocytes). However it is not necessary to isolate the cells and whole blood can be used instead, in which case the pharmaceutical composition comprises the oncolytic virus suspended in whole blood or whole blood containing leukocytes and/or platelets infected with the virus. Optionally the leukocytes or platelets are first isolated from whole blood, mixed or infected with the virus and then added back to the other blood components. In different embodiments of this invention the leukocytes are selected from monocytes, neutrophils and lymphocytes. In a more specific embodiment of this invention the leukocytes are tumor-infiltrating lymphocytes (TILs). In accordance with one embodiment of this invention the oncolytic virus utilized can be of low (lentogenic), moderate (mesogenic) or high (velogenic) virulence.

In an embodiment of this invention the virus is a clonal virus. Referring to the method or use in which the pharmaceutical composition utilized comprises leukocytes and oncolytic virus in suspension, in an embodiment of such method the ratio of plaque-forming units of the virus to number of leukocytes in the composition is at least 1:1. Generally it is preferred that the leukocytes be saturated with active virus particles. In the case of NDV saturation is achieved at a 200:1 ratio of plaque-forming units of the virus to number of leukocytes. Accordingly in an embodiment of this invention the virus is NDV and the ratio of plaque-forming units of the virus to number of leukocytes in the composition is from about 1:1 to about 200:1, and preferably is about 200:1. In the method or use described above in which the pharmaceutical composition utilized comprises cells infected with an oncolytic virus, in an embodiment of such method the infected cells are at least one-tenth of one percent (0.1%) of the total number of leukocytes and platelets in the composition, more preferably at least thirty percent and most preferably about one hundred percent. The virus utilized can be replication incompetent although preferably it is replication competent. In an embodiment of this invention the oncolytic virus is selected from the group consisting of a Newcastle Disease Virus (NDV), a Mumps Virus, a Measles Virus, a Vesicular Stomatitis Virus, a Para-influenza Virus, an Influenza Virus, an Adenovirus, a Herpes I Virus, a Vaccinia Virus, and a Reovirus. In a more specific embodiment a Newcastle Disease Virus strain of moderate virulence can be utilized. The skilled clinician can determine the optimal amount of the composition to be administered in each case. Typically, when the cells are leukocytes the effective amount is a daily dosage of the composition containing from 6×10⁶ to 6×10¹⁰ leukocytes per square meter of patient surface area, for example about 6×10⁷ leukocytes per square meter of patient surface area. When the cells are platelets the effective amount is typically a daily dosage of the composition containing from 10⁹ to 10¹¹ platelets per square meter of patient surface area, for example about 10¹¹ platelets per square meter of patient surface area.

The daily dosage of the composition can be administered to the subject in multiple administrations in the course of a single twenty-four hour period in which a portion of the daily dosage is administered at each administration. More preferably the daily dosage is administered in a single administration. In an embodiment of this invention the daily dosage of the composition is administered to the subject at a frequency of from one to seven times (i.e. on each of from one to seven days) in a one-week period. In accordance with this invention, any conventional route of administration is suitable for administering the pharmaceutical composition. For example, the composition can be administered intravenously, intratumorally, intraperitoneally or intravesicularly (kidneys). In the case of intravenous administration, it is convenient if the volume of the composition administered is from twenty-five milliliters to one liter. In the case of intratumoral administration it is convenient if the volume of composition administered is from one hundred microliters to ten milliliters per tumor mass. In the case of intraperitoneal administration, it is convenient if the volume of composition administered is up to two liters. In the case of intravesicular administration it is convenient if the volume of composition administered is up to seventy-five milliliters, preferably from fifty to sixty milliliters. Depending on the amount of pfus of virus and cells to be administered the concentration of the composition can be varied to achieve the desired volume. When the cancer is a solid tumor the composition can be administered by any of the routes given above, for example intravenously or intratumorally. When the cancer is other than a solid tumor (e.g. leukemia) the composition is not administered intratumorally and instead can be administered by the other routes given above, for example intravenously.

All references cited herein are all incorporated by reference herein, in their entirety, whether specifically incorporated or not. All publications, patents, patent applications, GenBank sequences and ATCC deposits, cited herein are hereby expressly incorporated by reference for all purposes. In particular, all nucleotide sequences, amino acid sequences, nucleic constructs, DNA vaccines, oncolytic virusus, methods of administration, particular orders of administration of DNA vaccines and agents, such as oncolytic viruses and cell therapies that are described in the patents, patent applications and other publications referred to herein or authored by one or more of the inventors of this application are specifically incorporated by reference herein. In case of conflict, the definitions within the instant application govern.

1. A method of inducing antitumor immune responses comprising: a) selecting a patient suffering from cancer; b) administering an immunogenic preparation capable of inducing an immune response towards tumor endothelium; and c) administering an oncolytic virus.

2. The method of claim 1, wherein the immunogenic preparation is comprised of endothelial progenitor cells cultured in a manner to endow the cells with ability to express antigens found on tumor endothelium.

3. The method of claim 1, wherein the oncolytic virus possesses ability to selectively home to the tumor and induce expression of molecules associated with tumor angiogenesis in a manner so as to stimulate immunity capable of targeting tumor vasculature.

4. The method of claim 1, wherein the immunogenic preparation capable of inducing an immune response towards tumor endothelium is ValloVax.

5. The method of claim 1, wherein the oncolytic virus is selected from a group of viruses consisting of: a) reovirus; b) herpes virus; c) New Castle Disease Virus; d) human papilloma virus, and e) vaccinia virus.

6. The method of claim 5, wherein the oncolytic virus produces an interferon response when administered systemically.

7. The method of claim 5, wherein the oncolytic virus produces an interferon response when administered intratumorally.

8. The method of claim 3, wherein the molecules resembling tumor vascular markers are selected from a group consisting of: TEM-1, CD105, VEGF-R, EGF-R, ROBO family members, PDGF-receptor, and angiopoietin receptor.

9. The method of claim 1, wherein the agent capable of inducing immune response towards tumor endothelial cells is derived from placental endothelial cells.

10. The method of claim 9, wherein the endothelial cells are cultured under hypoxia.

11. The method of claim 9, wherein the endothelial cells are cultured under acidic conditions.

12. The method of claim 9, wherein the endothelial cells are cultured with interferon gamma to augment expression of HLA antigens.

13. The method of claim 9, wherein the endothelial cells are endothelial progenitor cells.

14. The method of claim 9, wherein the endothelial cells are allogeneic to the recipient.

15. The method of claim 9, wherein the endothelial cells are xenogeneic to the recipient.
